(19) United States
(12) Patent Application Publication
(10) Pub. No.: US 2026/0105992 A1
(43) Pub. Date: Apr. 16, 2026

(54) FLUID IDENTIFICATION SYSTEM AND METHOD

(71) Applicant: INDUSTRIAL TECHNOLOGY RESEARCH INSTITUTE, Hsinchu (TW)

(72) Inventors: Yu-Tai LI, Taichung City (TW); Yu Hsiang LO, New Taipei City (TW); Yu-Hsiang HUANG, New Taipei City (TW)

(73) Assignee: INDUSTRIAL TECHNOLOGY RESEARCH INSTITUTE, Hsinchu (TW)

(21) Appl. No.: 18/986,533

(22) Filed: Dec. 18, 2024

(30) Foreign Application Priority Data
Oct. 16, 2024 (TW) 113139342

(51) Int. Cl. G16C 20/20; G01N 21/05; G01N 21/3577; G01N 21/3581; G01N 21/65; G16C 20/70

(52) U.S. Cl. CPC G16C 20/20; G01N 21/05; G01N 21/3577; G01N 21/3581; G01N 21/65; G16C 20/70; G01N 2201/121; G01N 2201/129

(57) ABSTRACT

The present disclosure provides a fluid identification system and method. The fluid identification system includes at least one fluid carrying device and a non-transitory computer-readable medium. The at least one fluid carrying device is configured to carry test fluid. The non-transitory computer-readable medium includes at least one computer-executable program, wherein steps are performed when the at least one computer-executable program is executed by a processor, and the steps comprise: obtaining a Raman spectrum and a Terahertz spectrum corresponding to same measuring time, removing a background spectrum from the Raman spectrum to generate a corrected Raman spectrum, and using an identification model based on the corrected Raman spectrum and the Terahertz spectrum to obtain a subject type of the test fluid.

### CROSS-REFERENCE TO RELATED APPLICATIONS

This non-provisional application claims priority under 35 U.S.C. § 119(a) on Patent Application No(s). 113139342 filed in Republic of China (Taiwan) on Oct. 16, 2024, the entire contents of which are hereby incorporated by reference.

### BACKGROUND

### 1. Technical Field

This disclosure relates to a fluid identification system and method.

### 2. Related Art

Currently, the testing in the fluid and biopharmaceutical industries still primarily relies on offline analysis methods, which typically require human resources and a certain level of technical expertise. In existing Raman measurement technology, the ability to accurately analyze mixture components in non-contact measurements is still undergoing continuous improvement.

Additionally, when analyzing test fluid with a single measurement instrument, the measurement outcome may vary depending on the instrument's sensitivity and resolution, especially for materials with lower reactivity.

### SUMMARY

According to one or more embodiment of this disclosure, a fluid identification system includes at least one fluid carrying device and a non-transitory computer-readable medium. The at least one fluid carrying device is configured to carry test fluid. The non-transitory computer-readable medium includes at least one computer-executable program, wherein steps are performed when the at least one computer-executable program is executed by a processor, and the steps comprise: obtaining a Raman spectrum and a Terahertz spectrum corresponding to same measuring time, removing a background spectrum from the Raman spectrum to generate a corrected Raman spectrum, and using an identification model based on the corrected Raman spectrum and the Terahertz spectrum to obtain a subject type of the test fluid.

According to one or more embodiment of this disclosure, a fluid identification method, performed by a processor, includes: obtaining a Raman spectrum corresponding to measuring time from a Raman measuring device; obtaining a Terahertz spectrum corresponding to the measuring time from a Terahertz measuring device; removing a background spectrum from the Raman spectrum to generate a corrected Raman spectrum; and using an identification model based on the corrected Raman spectrum and the Terahertz spectrum to obtain a subject type of a test fluid.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a block diagram illustrating a fluid identification system according to an embodiment of the present application;

FIG. 2 is an exploded diagram illustrating a fluid carrying device according to an embodiment of the present disclosure;

FIG. 3 is an exploded diagram illustrating a fluid carrying device according to another embodiment of the present disclosure;

FIG. 4 is a side view of a fluid carrying device according to yet another embodiment of the present disclosure;

FIG. 5 is a schematic diagram illustrating fluid carrying devices connected in series according to an embodiment of the present disclosure;

FIG. 6 is a flow chart illustrating a fluid identification method according to an embodiment of the present disclosure; and

FIG. 7 shows an exemplary diagram of curves of the Raman spectrum, the background spectrum and the corrected Raman spectrum.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the present invention. The following embodiments further illustrate various aspects of the present invention, but are not meant to limit the scope of the present invention.

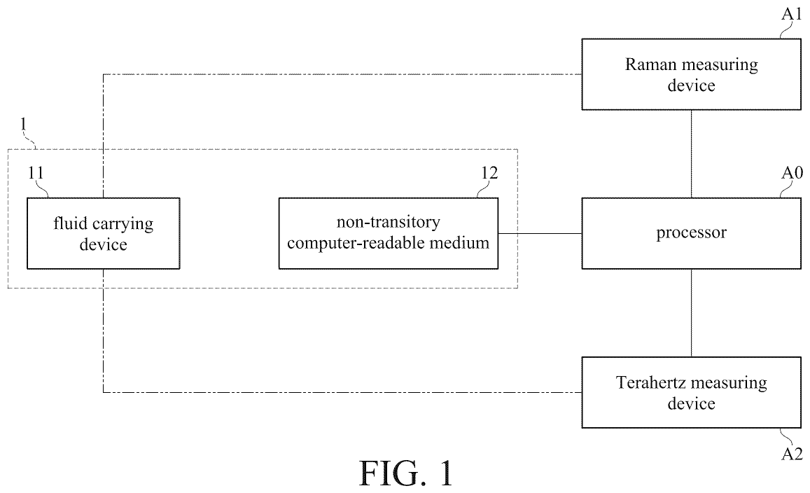

Please refer to FIG. 1, wherein FIG. 1 is a block diagram illustrating a fluid identification system according to an embodiment of the present application. As shown in FIG. 1, the fluid identification system 1 includes at least one fluid carrying device 11 and a non-transitory computer-readable medium 12. FIG. 1 shows one fluid carrying device 11, but the fluid identification system 1 may also be a plurality of fluid carrying devices 11.

The fluid carrying device 11 may be a light-transmissive fluid carrier. The fluid carrying device 11 is configured to carry (hold) test fluid, and be exposed to a Raman measuring device A1 and a Terahertz measuring device A2, which is exemplarily shown in FIG. 1 with two dotted lines. The test fluid may be a viable cell solution and/or a liquid containing multiple mixtures, the present disclosure does not limit the type of the test fluid.

The non-transitory computer-readable medium 12 is in signal communication with or electrically connected to the processor A0. The non-transitory computer-readable medium 12 comprises at least one computer-executable program, and a plurality of steps are performed when the at least one computer-executable program is executed by the processor A0. The steps include the fluid identification method described below. The steps are used to generate an analysis result of the test fluid according to the measurement results of the Raman measuring device A1 and the Terahertz measuring device A2. Further, the processor A0 used to execute the computer-executable program of the non-transitory computer-readable medium 12 may be in communication with the Raman measuring device A1 and the Terahertz measuring device A2, and when the computer-executable program is executed by the processor A0, the implemented steps may include controlling the Raman measuring device A1 and the Terahertz measuring device A2 to emit measuring signals simultaneously, and the measuring signals may be emitted towards the fluid carrying device 11. The non-transitory computer-readable medium 12 may be a hard disk, optical disk, USB drive, magnetic tape, flash memory, read-only memory, or a database accessible over the internet. The processor A0 may be a central processing unit (CPU), graphics processing unit (GPU), microcontroller, programmable logic controller (PLC), or other processors with signal processing functions.

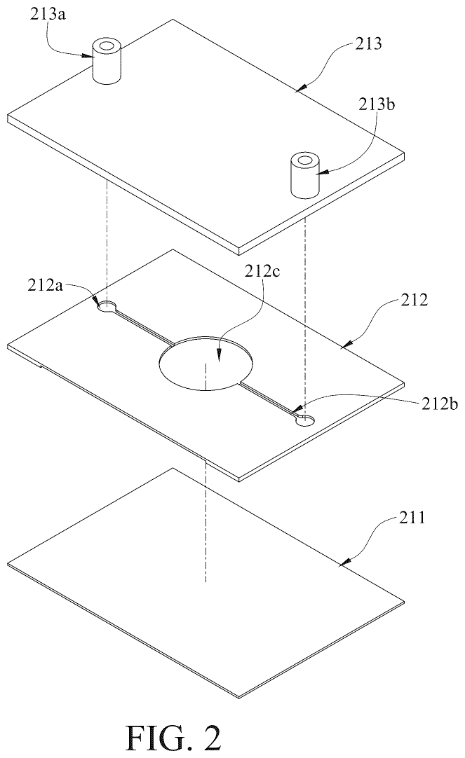

Please refer to FIG. 2, wherein FIG. 2 is an exploded diagram illustrating a fluid carrying device according to an embodiment of the present disclosure. As shown in FIG. 2, the fluid carrying device 21 may include a silicon substrate 211, a flow channel bottom cover 212 and a flow channel top cover 213.

The flow channel bottom cover 212 is disposed on the silicon substrate 211. The flow channel bottom cover 212 includes an inlet slot 212a, an outlet slot 212b and a fluid containment region 212c in communication with the inlet slot 212a and the outlet slot 212b. The fluid containment region 212c is configured to hold (accommodate) the test fluid. When the test fluid held in the fluid containment region 212c is a viable cell solution, the fluid containment region 212c may be used as a culture zone for viable cell differentiation. The inlet slot 212a and the outlet slot 212b each includes a circular hole, with a linear slit extending from the circular hole toward one side of the fluid containment region 212c, and the slit is in communication with the fluid containment region 212c.

The flow channel top cover 213 is disposed on the flow channel bottom cover 212. The flow channel top cover 213 includes a flow channel inlet 213a and a flow channel outlet 213b, wherein the flow channel inlet 213a is in communication with the inlet slot 212a, and the flow channel outlet 213b is in communication with the outlet slot 212b. The flow channel inlet 213a and the flow channel outlet 213b may each be implemented in the form of conductive tube. The flow channel inlet 213a is aligned with the inlet slot 212a, and the flow channel outlet 213b is aligned with the outlet slot 212b. An aperture of the flow channel inlet 213a is preferably the same as an aperture of the inlet slot 212a, and an aperture of the flow channel outlet 213b is preferably the same as an aperture of the circular hole of the outlet slot 212b.

In an embodiment, the flow channel bottom cover 212 and the flow channel top cover 213 are preferably made of material that allows the measuring signal emitted by the Raman measuring device A1 to pass through. Further, the flow channel bottom cover 212 and the flow channel top cover 213 may both be made of polydimethylsiloxane (PDMS) material, glass material or quartz material.

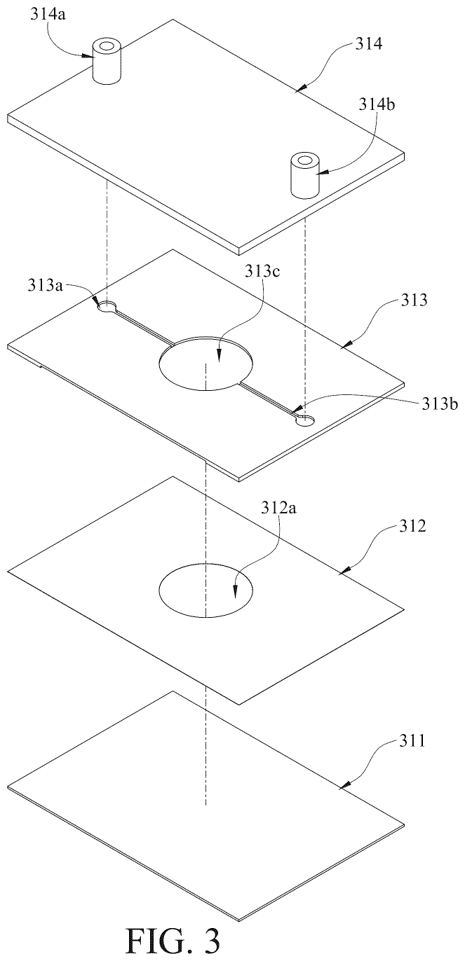

Please refer to FIG. 3, wherein FIG. 3 is an exploded diagram illustrating a fluid carrying device according to another embodiment of the present disclosure. As shown in FIG. 3, the fluid carrying device 31 may include a silicon substrate 311, a biocompatible gel 312, a flow channel bottom cover 313 and a flow channel top cover 314.

The flow channel bottom cover 313 includes an inlet slot 313a, an outlet slot 313b and a fluid containment region 313c in communication with the inlet slot 313a and the outlet slot 313b. The fluid containment region 313c is configured to hold (accommodate) the test fluid. The flow channel top cover 314 is disposed on the flow channel bottom cover 313. The flow channel top cover 314 includes a flow channel inlet 314a and a flow channel outlet 314b, wherein the flow channel inlet 314a is in communication with the inlet slot 313a and the flow channel outlet 314b is in communication with the outlet slot 313b.

The silicon substrate 311, the flow channel bottom cover 313 and the flow channel top cover 314 may be the same as the silicon substrate 211, the flow channel bottom cover 212 and the flow channel top cover 213 shown in FIG. 2, respectively, their details are not repeated herein.

The biocompatible gel 312 is disposed between the flow channel bottom cover 313 and the silicon substrate 311. The biocompatible gel 312 includes a hole 312a. The hole 312a is in communication with the fluid containment region 313c, and a dimension (aperture) of the hole 312a may be the same as a dimension (aperture) of the fluid containment region 313c. Further, the hole 312a and the fluid containment region 313c may have the same dimension and the same profile. Therefore, the test fluid may stay in the space formed by the hole 312a and the fluid containment region 313c through the silicon substrate 311 and the flow channel top cover 314.

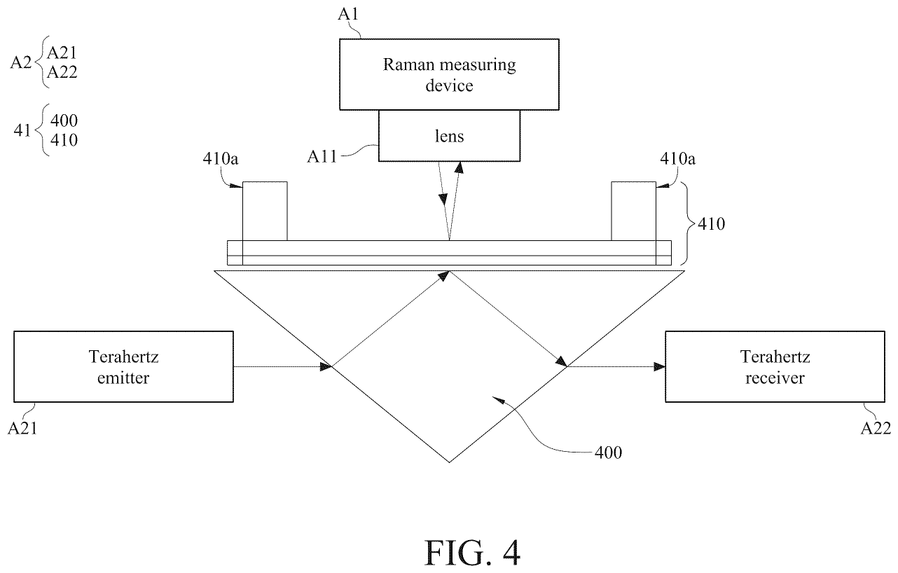

Please refer to FIG. 4, wherein FIG. 4 is a side view of a fluid carrying device according to yet another embodiment of the present disclosure. As shown in FIG. 4, the fluid carrying device 41 includes a silicon prism 400 and a carrying part 410. The carrying part 410 is disposed on the silicon prism 400. The carrying part 410 may be implemented as the fluid carrying device 21 shown in FIG. 2 or the fluid carrying device 21 shown in FIG. 3. The Raman measuring device A1 may include a lens All, and the carrying part 410 may be disposed below the Raman measuring device A1. The lens All is configured to emit measuring signal towards the carrying part 410 and receive a reflected signal from the carrying part 410.

The silicon prism 400 may be disposed below the silicon substrate of the carrying part 410. That is, the silicon prism 400 may be disposed at a side of the silicon substrate opposite to the flow channel bottom cover. As shown in FIG. 4, the Terahertz measuring device A2 may include a Terahertz emitter A21 and a Terahertz receiver A22. The fluid carrying device 41 may be disposed between the Terahertz emitter A21 and the Terahertz receiver A22. The Terahertz emitter A21 emits the measuring signal towards the silicon prism 400, and the Terahertz receiver A22 receives the reflected signal from the silicon prism 400. Further, the silicon prism 400 may allow the measuring signal emitted by the Terahertz emitter A21 to undergo total internal reflection.

In the fluid carrying device of one or more embodiments described above, the surface of the silicon substrate facing the flow channel bottom cover may include a plurality of periodic patterns.

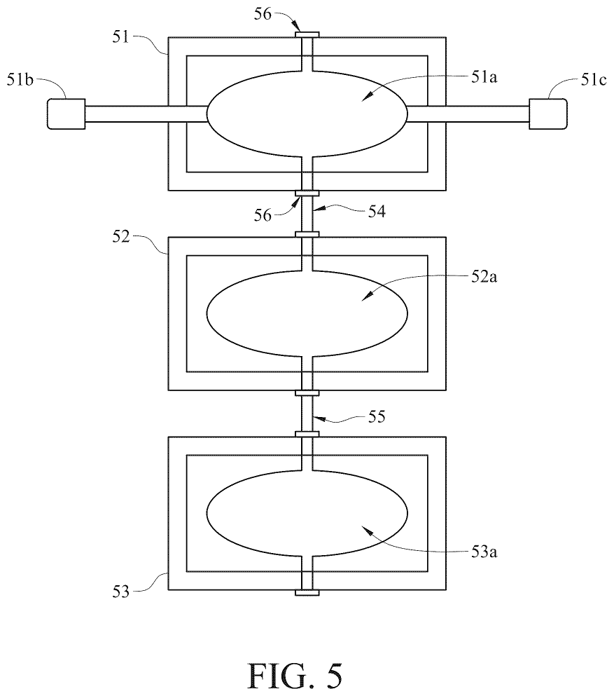

Please refer to FIG. 5, wherein FIG. 5 is a schematic diagram illustrating fluid carrying devices connected in series according to an embodiment of the present disclosure. As shown in FIG. 5, the at least one fluid carrying device described above may include a first fluid carrying device 51, a second fluid carrying device 52 and a third fluid carrying device 53. Each of the first fluid carrying device 51, the second fluid carrying device 52 and the third fluid carrying device 53 may be implemented as the fluid carrying device shown in any one of FIG. 2 to FIG. 4. It should be noted that FIG. 5 exemplarily shows three fluid carrying devices connected in series, but the number of connected fluid carrying devices may also be two or more than three, the present disclosure is not limited thereto.

The first fluid carrying device 51 includes a fluid containment region 51a, the second fluid carrying device 52 includes a fluid containment region 52a, and the third fluid carrying device 53 includes a fluid containment region 53a. The fluid containment region 51a, the fluid containment region 52a and the fluid containment region 53a may be configured to carry the same or different test fluid.

One of the flow channel inlet and the flow channel outlet of each of the first fluid carrying device 51, the second fluid carrying device 52 and the third fluid carrying device 53 is in communication with another one of the flow channel inlet and the flow channel outlet of an adjacent one of the first fluid carrying device 51, the second fluid carrying device 52 and the third fluid carrying device 53.

Specifically, the flow channel outlet of the first fluid carrying device 51 is in communication with the flow channel inlet of the second fluid carrying device 52, and the flow channel outlet of the second fluid carrying device 52 is in communication with the flow channel inlet of the third fluid carrying device 53.

The flow channel outlet of the first fluid carrying device 51 and the flow channel inlet of the second fluid carrying device 52 may be connected with each other through a first connecting tube 54 therebetween, and the flow channel outlet of the second fluid carrying device 52 and the flow channel inlet of the third fluid carrying device 53 may be connected with each other through a second connecting tube 55 therebetween.

Each of the flow channel inlet of the first fluid carrying device 51 and the flow channel outlet of the third fluid carrying device 53 may be disposed with a soft plug 56, to avoid the test fluid to flow out from the flow channel inlet of the first fluid carrying device 51 and the flow channel outlet of the third fluid carrying device 53. In an embodiment, the flow channel inlet and the flow channel outlet of each of the first fluid carrying device 51, the second fluid carrying device 52 and the third fluid carrying device 53 may be disposed with the soft plug 56, to avoid the test fluid of any one of the first fluid carrying device 51, the second fluid carrying device 52 and the third fluid carrying device 53 to flow into another one of the first fluid carrying device 51, the second fluid carrying device 52 and the third fluid carrying device 53.

Further, the first fluid carrying device 51 may further include a first connector 51b and a second connector 51c. The first connector 51b and the second connector 51c are in communication with the fluid containment region 51a of the first fluid carrying device 51. One of the first connector 51b and the second connector 51c may be used for the test fluid to flow into the fluid containment region 51a, and another one of the first connector 51b and the second connector 51c may be used for the test fluid to flow out from the fluid containment region 51a.

In the embodiment of FIG. 5, the test fluid may flow into the first fluid carrying device 51 through one of the first connector 51b and the second connector 51c, and then flow into the second fluid carrying device 52 and the third fluid carrying device 53 through the first connecting tube 54 and the second connecting tube 55. Accordingly, multiple fluid carrying devices may be used for the identification of the same or different test fluids, thereby improving measurement efficiency.

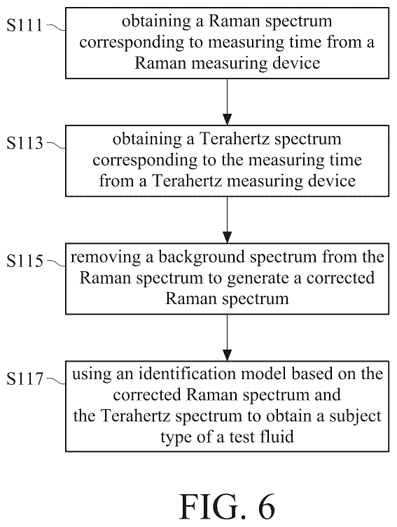

Please refer to FIG. 6, wherein FIG. 6 is a flow chart illustrating a fluid identification method according to an embodiment of the present disclosure. The fluid identification method is performed by the processor, the processor executes the computer-executable program stored by the non-transitory computer-readable medium described above to implement the fluid identification method. As shown in FIG. 6, the fluid identification method includes: step S111: obtaining a Raman spectrum corresponding to measuring time from a Raman measuring device; step S113: obtaining a Terahertz spectrum corresponding to the measuring time from a Terahertz measuring device; step S115: removing a background spectrum from the Raman spectrum to generate a corrected Raman spectrum; and step S117: using an identification model based on the corrected Raman spectrum and the Terahertz spectrum to obtain a subject type of a test fluid. The present disclosure does not limit the sequence of performing step S111 and step S113, and S113 may be performed before S111 or performed at the same time as S111.

In step S111, the processor obtains the Raman spectrum from the Raman measuring device, and the Raman spectrum is generated by the Raman measuring device emitting the measuring signal towards the fluid carrying device as described above. In step S113, the processor obtains the Terahertz spectrum from the Terahertz measuring device, and the Terahertz spectrum is generated by the Terahertz measuring device emitting the measuring signal towards the same fluid carrying device. The processor may obtain the Raman spectrum from the Raman measuring device and obtain the Terahertz spectrum from the Terahertz measuring device at the same time or separately, but the Raman spectrum and the Terahertz spectrum corresponding to the same measuring time are generated by the Raman measuring device and the Terahertz measuring device emitting the measuring signals towards the same fluid carrying device at the same time. In an embodiment, the processor may extract a part of each of the Raman spectrum and the Terahertz spectrum corresponding to the same measuring time according to the timestamp and measuring duration of each of the Raman spectrum and the Terahertz spectrum, and use the extracted parts as the Raman spectrum and the Terahertz spectrum used in the following steps.

In step S115, the processor removes the background spectrum from the Raman spectrum to generate the corrected Raman spectrum. Accordingly, the subsequent analysis of the test fluid may not be affected by the background spectrum.

In step S117, the processor inputs the corrected Raman spectrum and the Terahertz spectrum into the identification model to obtain the subject type output by the identification model. The subject type may indicate the components of the test fluid. The identification model may include at least one of a linear discriminant analysis model, a kernel logistic regression model and a subspace k-nearest neighbor model.

In view of the above, the fluid identification system and method according to one or more embodiments of the present disclosure may provide a non-contact, dual-spectral analysis technique, which may be used for online, real-time, and continuous identification. Furthermore, in the dual-spectral analysis technique, Raman spectrum and Terahertz spectrum may be used complementarily to obtain more accurate identification result of the test fluid.

In a detailed embodiment of step S115, the processor may perform asymmetrically reweighted penalized least square (arPLS) algorithm on the Raman spectrum to obtain the corrected Raman spectrum. The processor may perform the arPLS algorithm through equation (1) below, wherein x is the raw Raman spectrum, z is the background spectrum, and D is the difference matrix, W is diagonal matrix of the weight vector, λ is the smoothness coefficient. As the smoothness coefficient decreases, the background spectrum becomes closer to the Raman spectrum signal; conversely, as the smoothness coefficient increases, the background spectrum becomes smoother.

$$z = {( W + λ {D}^{T} D )}^{- 1} Wx \qquad equation ( 1 )$$

Further, the processor may use equation (2) below to perform iteration for the background spectrum to converge to a preset value as the optimal solution, wherein w is the weight vector of the diagonal matrix W described above, t is the current calculation number (i.e., the iteration count), and r is the target convergence ratio. The target convergence ratio may be set in the range of 10⁻¹ to 10⁻⁶, but the present dislcosure is not limited thereto. The processor may set an upper limit for the iteration count.

$$\frac{\vert {w}^{t} - {w}^{t + 1} \vert}{\vert {w}^{t} \vert} < r \qquad equation ( 2 )$$

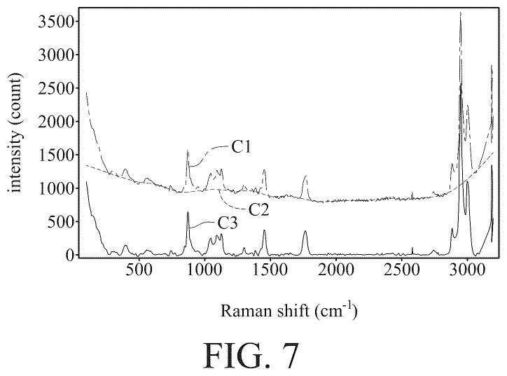

Please refer to FIG. 7, wherein FIG. 7 shows an exemplary diagram of curves of the Raman spectrum, the background spectrum and the corrected Raman spectrum. As shown in FIG. 7, the processor obtains the Raman spectrum C1 from the Raman measuring device, determines the background spectrum C2 corresponding to the Raman spectrum C1, and removes the background spectrum C2 from the Raman spectrum C1 to generate the corrected Raman spectrum C3.

In an embodiment, the fluid identification method may further include the processor using a plurality of Raman training spectrums and a plurality of Terahertz training spectrums corresponding to the subject type to perform training to obtain the identification model described in step S117 in FIG. 6. Specifically, each subject type may have corresponding Raman training spectrums and Terahertz training spectrums, the processor may use the Raman training spectrums and the Terahertz training spectrums to train at least one of a linear discriminant analysis model, a kernel logistic regression model and a subspace k-nearest neighbor model to generate the identification model, wherein the Raman training spectrums may be spectrums with background spectrums already removed.

Further, in the step of using the Raman training spectrums and the Terahertz training spectrums corresponding to the subject type to perform training to obtain the identification model, the processor may use the Raman training spectrums and the Terahertz training spectrums to train a plurality of candidate models, and select one of the candidate models with a highest accuracy as the identification model. In other words, the processor may use the Raman training spectrums and the Terahertz training spectrums to perform training to obtain the candidate models, use validation dataset to verify the trained candidate models, and select one of the candidate models with the highest accuracy as the identification model used in step S117 in FIG. 6.

In addition, in the step of using the Raman training spectrums and the Terahertz training spectrums corresponding to the subject type to perform training to obtain the identification model, the processor may further use the Raman training spectrums to perform training to generate a first sub-model, use the Terahertz training spectrums to perform training to generate a second sub-model, and fuse the first sub-model and the second sub-model into the identification model. Accordingly, the identification model may be configured to determine the subject type according to the Raman spectrum and the Terahertz spectrum. Each of the first sub-model and the second sub-model may be at least one of a linear discriminant analysis model, a kernel logistic regression model and a subspace k-nearest neighbor model.

In an implementation, steps S111, S113, S115 and S117 described above may be performed by the processor, and the training of the identification model may be performed by another processor, and said another processor stores the trained identification model into the non-transitory computer-readable medium shown in FIG. 1. In another implementation, steps S111, S113, S115 and S117 described above as well as the steps of training the identification model may be one or more computer-executable programs stored by the non-transitory computer-readable medium and executed by the same processor, and the same processor may store the trained identification model into the non-transitory computer-readable medium shown in FIG. 1.

In view of the above, the fluid identification system and method according to one or more embodiments of the present disclosure may provide a non-contact, dual-spectral analysis technique, which may be used for online, real-time, and continuous identification. Furthermore, in the dual-spectral analysis technique, Raman spectrum and Terahertz spectrum may be used complementarily to obtain more accurate identification result of the test fluid. Further, by removing the background spectrum from the Raman spectrum, the subsequent analysis of the test fluid may not be affected by the background spectrum. In addition, by connecting fluid carrying devices in series, the fluid carrying devices may be used for the identification of the same or different test fluids, thereby improving measurement efficiency.

What is claimed is:

1. A fluid identification system, comprising:
at least one fluid carrying device configured to carry test fluid; and
a non-transitory computer-readable medium comprising at least one computer-executable program, wherein a plurality of steps are performed when the at least one computer-executable program is executed by a processor, and the plurality of steps comprise: obtaining a Raman spectrum and a Terahertz spectrum corresponding to same measuring time, removing a background spectrum from the Raman spectrum to generate a corrected Raman spectrum, and using an identification model based on the corrected Raman spectrum and the Terahertz spectrum to obtain a subject type of the test fluid.

2. The fluid identification system according to claim 1, wherein removing the background spectrum from the Raman spectrum to generate the corrected Raman spectrum comprises: performing asymmetrically reweighted penalized least square algorithm on the Raman spectrum to obtain the corrected Raman spectrum.

3. The fluid identification system according to claim 1, wherein the plurality of steps further comprise: using a plurality of Raman training spectrums and a plurality of Terahertz training spectrums corresponding to the subject type to perform training to obtain the identification model.

4. The fluid identification system according to claim 3, wherein using the plurality of Raman training spectrums and the plurality of Terahertz training spectrums corresponding to the subject type to perform training to obtain the identification model comprises:
using the plurality of Raman training spectrums to perform training to generate a first sub-model, using the plurality of Terahertz training spectrums to perform training to generate a second sub-model, and fusing the first sub-model and the second sub-model into the identification model.

5. The fluid identification system according to claim 3, wherein using the plurality of Raman training spectrums and the plurality of Terahertz training spectrums corresponding to the subject type to perform training to obtain the identification model comprises: using the plurality of Raman training spectrums and the plurality of Terahertz training spectrums to train a plurality of candidate models, and selecting one of the plurality of candidate models with a highest accuracy as the identification model.

6. The fluid identification system according to claim 1, wherein the identification model comprises: at least one of a linear discriminant analysis model, a kernel logistic regression model and a subspace k-nearest neighbor model.

7. The fluid identification system according to claim 1, wherein the at least one fluid carrying device comprises:
a silicon substrate;
a flow channel bottom cover disposed on the silicon substrate, the flow channel bottom cover comprising an inlet slot, an outlet slot, and a fluid containment region in communication with the inlet slot and the outlet slot, the fluid containment region is configured to hold the test fluid; and
a flow channel top cover disposed on the flow channel bottom cover, the flow channel top cover comprising a flow channel inlet and a flow channel outlet, wherein the flow channel inlet connects the inlet slot, and the flow channel outlet connects the outlet slot.

8. The fluid identification system according to claim 7, wherein the flow channel top cover comprises polydimethylsiloxane material.

9. The fluid identification system according to claim 7, wherein the at least one fluid carrying device comprises a plurality of fluid carrying devices, one of the flow channel inlet and the flow channel outlet of each of the plurality of fluid carrying devices connects another one of the flow channel inlet and the flow channel outlet of an adjacent one of the plurality of fluid carrying devices.

10. The fluid identification system according to claim 7, wherein the at least one fluid carrying device further comprises:
a biocompatible gel disposed between the flow channel bottom cover and the silicon substrate.

11. The fluid identification system according to claim 10, wherein the biocompatible gel comprises a hole in communication with the fluid containment region, wherein a dimension of the hole is the same as a dimension of the fluid containment region.

12. The fluid identification system according to claim 7, wherein the at least one fluid carrying device further comprises:
a silicon prism disposed under the silicon substrate.

13. The fluid identification system according to claim 7, wherein a surface of the silicon substrate facing the flow channel bottom cover comprises a plurality of periodic patterns.

14. A fluid identification method, performed by a processor, comprising:
obtaining a Raman spectrum corresponding to measuring time from a Raman measuring device;
obtaining a Terahertz spectrum corresponding to the measuring time from a Terahertz measuring device;
removing a background spectrum from the Raman spectrum to generate a corrected Raman spectrum; and
using an identification model based on the corrected Raman spectrum and the Terahertz spectrum to obtain a subject type of a test fluid.

15. The fluid identification method according to claim 14, wherein removing the background spectrum from the Raman spectrum to generate the corrected Raman spectrum comprises:
performing asymmetrically reweighted penalized least square algorithm on the Raman spectrum to obtain the corrected Raman spectrum.

16. The fluid identification method according to claim 14, further comprising:
using a plurality of Raman training spectrums and a plurality of Terahertz training spectrums corresponding to the subject type to perform training to obtain the identification model.

17. The fluid identification method according to claim 16, wherein using the plurality of Raman training spectrums and the plurality of Terahertz training spectrums corresponding to the subject type to perform training to obtain the identification model comprises:
using the plurality of Raman training spectrums to perform training to generate a first sub-model, using the plurality of Terahertz training spectrums to perform training to generate a second sub-model, and fusing the first sub-model and the second sub-model into the identification model.

18. The fluid identification method according to claim 16, wherein using the plurality of Raman training spectrums and the plurality of Terahertz training spectrums corresponding to the subject type to perform training to obtain the identification model comprises:
using the plurality of Raman training spectrums and the plurality of Terahertz training spectrums to train a plurality of candidate models, and selecting one of the plurality of candidate models with a highest accuracy as the identification model.

19. The fluid identification method according to claim 14, wherein the identification model comprises: at least one of a linear discriminant analysis model, a kernel logistic regression model and a subspace k-nearest neighbor model.
